(19) United States
(12) Patent Application Publication
(10) Pub. No.: US 2006/0075521 A1
(43) Pub. Date: Apr. 6, 2006

(54) Method of producing and screening antibodies produced in transgenic plants

(76) Inventor: Carsten Meier, Hjortholms Alle (DK)

Correspondence Address: Gabor L Szekeres;Law Offices Of Gabor L. Szekeres, Suite 112, 8141 Kaiser Boulevard, Anaheim, CA, 92808, US

(21) Appl. No.: 10/528,208

(22) PCT Filed: Sep. 19, 2003
(86) PCT No.: PCT/IB03/04056

§ 371 Date: Sep. 15, 2005

(30) Foreign Application Priority Data
Sep. 20, 2002 (GB) 0221940.0

(51) Int. Cl. A01H 1/00; C07H 21/04; C12N 15/82; C12N 5/04; C07K 16/18

(52) U.S. Cl. 800/288; 530/387.1; 435/419; 435/468; 536/23.53

(57) ABSTRACT

The present invention relates to the production of antibodies in transgenic plants and screening of said antibodies for binding to antigens. The invention provides a method by which a large number of antibodies with variations in the multivariable regions are produced and screened for binding to antigens. The invention is particularly useful for the production of antibodies to antigens which may be present in different variants such as for example HIV-1 and A- or B-influenza viruses.

The present invention relates to a method of producing antibodies in transgenic plants and a method of screening such antibodies.

AIDS is caused by the human immunodeficiency virus-1 (HIV-1) and is one of the major infectious diseases affecting more than 40 million people worldwide (UNIAIDS estimation, 2001). Both industrialized world and low-income countries are concerned. Despite the efforts undertaken by researchers during the last 20 years, only few treatments are available to date. Moreover, the cost of the highly active anti-retroviral therapy is prohibitive in non-industrialized countries. The HIV-1 virus employs a multitude of schemes to generate variants, such as accumulation of base substitutions, insertions and/or deletions within its genome, addition and/or loss of glycosylation sites in the envelope protein, as well as recombination. This ability to mutate in infected individuals is one of the main reasons why the virus escapes natural defences before infected patients become immuno-compromised.

The molecular structure of human antibodies that are specific for HIV-1 are of major interest as AIDS research progresses toward passive immunotherapeutics in the maintenance and prevention of infection. In recent years a number of human, HIV-specific hybridomas and Epstein-Barr virus (EBV)-transformed B cell lines, as well as a combinatorial library, have been developed and characterized at the molecular level. These sources have provided valuable information on the immunoglobulin heavy- and light-chain variable-region gene usage and the extent and appearance of somatic mutation in a disease where the immune system is under constant stimulation over a long period of time.

Despite major advances in in vitro synthesis of monoclonal antibodies (mAB), such as the hybridoma method, production of purified antibody preparations in commercially acceptable quantities remains a challenge. In particular, existing commercial methods require expensive colonies of laboratory animals or large-scale cell culture facilities. Moreover, hybridomas involve the use of EBV, murine myeloma fusion partners, and may also entail bovine component during transformation and adaptation to serum-free media. Therefore, purification of recombinant antibodies from animals or from animal cell culture relies on the ability to remove potentially contagious agents, especially in light of the recent bovine epidemic of spongiform encephalopathy (BSE) and the related human disease entity, variant Creutzfeld-Jacob disease (Will et al., 1996). Additionally, such purification requires the preservation of the conformational integrity and biological activity of the mAB.

Since the early description of mAB (Kohler and Milstein 1975) there has been great interest in their use in human serotherapy. However, several problems have hindered their adoption, including the antiglobulin response, which is observed with antibodies produced in rodents in prolonged human therapy. This antiglobulin response is due to sequence divergence between human and rodent antibody molecules (Kabat et al., 1991), such that injection of rodent mAB into patients usually elicits an antiglobulin response at 8-12 days with a peak at 20-30 days (Isaacs et al., 1990). The antiglobulin response therefore precludes treatment beyond 10 days, and the rapid onset of a secondary response prevents further treatment.

As alternative to rodents, antibodies could be supplied by immunized volunteers. However, deliberate immunization of a hitherto non-immunized individual presents both practical and ethical problems (Power et al., 1995).

Protein engineering using various recombinant DNA technologies offers potential solutions to some of these problems. One such approach is the production of antibodies in plants (Richter et al., 2000, Belanger et al., 2000; De Jaeger et al., 2000). Transgenic plants represent a cheap and safe alternative to cell culture systems for producing human proteins. Protein processing is well conserved among plant and mammalian cells. For example, both mammalian and plant proteins undergo post translational modifications such as glycosylation, complex folding or multimeric assembly. Since transgenic plants have been shown to express correctly folded, full-length antibody (Hiatt et al., 1989), the opportunity offered for large-scale production of cheap recombinant antibodies or immunoglobulin-derived fragments lead to potential diagnostic and therapeutic applications (Giddings et al., 2000; Fisher & Emans, 2001). Therapeutical immunoglobulins produced in transgenic plants have so far been mostly targeted against mucosa-associated diseases (Ma et al., 1998; Verch et al., 1998; Zeitlin et al., 1998). Recently, production of an antibody targeted against an antigen present in the blood stream was reported (Bouquin et al., 2002).

Potential advantages of plant-derived antibodies, referred here as “plantibodies”, include
(i) The capacity for producing large amounts of the desired antibody.
(ii) Plant cells, in contrast to human cells, are not natural hosts for human pathogens; i.e. the risk of contamination with vira or prions from plants is essentially nonexistent
(iii) Transgenic plants expressing the gene of interest are easily generated from various species using well-established techniques. This is in contrast to animal/human cell cultures that require fusion to myeloma partners to immortalize valuable cell lines during creation of hybridomas.
(iv) Transgenic plants can be stored as seeds, an extremely cheap and stable method of storage.

The present invention provides a solution to the problems associated with the prior art by generating human antibody libraries, targeted against infectious agents, in transgenic plants. Specific examples include antibodies targeted against HIV-1 and A- or B-influenza viruses.

An “Immunoglobulin” is an antibody. Antibodies are composed of constant regions (Fc) that determine the effector function of the antibody and the antigen binding domains (Fab) which comprise a unique set of complementarity determining regions (CDRs). The role of antibodies is to bind to antigens, thereby making them more visible to the immune system. Antigen-antibody (Ag-Ab) complexes can bind to Fc receptor on phagocytic cells to allow efficient digestion of harmful pathogens. Fc receptor internalisation also allows efficient antigen presentation to stimulate T cell responses. Previous studies have shown that Fc receptor internalisation of Ag-Ab complexes is 1,000 fold more efficient than pinocytosis for stimulation of helperT cell responses. More recently, it has been shown that Fc receptor internalisation of antigen-antibody complexes by dendritic cells also allows more efficient processing of antigen into class I and class II presentation pathways to allow stimulation of not only helper but also cytotoxic T cell responses (Regnault et al., (1999) J exp Med. 189: 371-380). Furthermore, Fc receptor internalisation activates dendritic cells to express co-stimulatory molecules essential for priming naïve responses. Fc receptor internalisation is a consequence of receptor cross linking by antigen/antibody complexes and previous studies have shown that Ab alone cannot mediate this effect.

The term “immunoglobulin” also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses; fragments which comprise an antigen binding domain such as Fab, scFv, Fv, dAb, Fd; and diabodies.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv dimers (PCT/US92/09965) and (ix) “diabodies”, multivalent or multispecific fragments constructed by gene fusion (WO94/13804; P. Hollinger et al., Proc. Natl. Acad. Sci. USA 90:6444-6448 (1993)).

Each immunoglobulin molecule is comprised of four polypeptide chains, two identical light chains and two identical heavy chains, held together by interchain disulphide bonds. Each polypeptide chain contains a constant domain and a variable domain. The variable domain contains three hypervariable sequences, forming the “multivariable region” which is involved in the recognition of the antigen. The coding sequence of the multivariable regions can be altered by exchanging, inserting or deleting one or more nucleotides as compared to the original said immunoglobulin sequence. This provides a large number of combinations in the variable domain, so producing antibodies which can bind to a large number of antigens.

Thus, in the first aspect the present invention provides a nucleic acid construct comprising:
(i) a sequence encoding an immunoglobulin heavy chain,
(ii) a sequence encoding an immunoglobulin light chain, and
(iii) one or more promoters capable of controlling expression of both sequences in a plant,
   wherein both said sequences contain a multivariable region which have been altered by exchanging, inserting or deleting one or more nucleotides as compared to the original said immunoglobulin sequence. The nucleic acid can be either DNA or RNA.

The promoter or promoters may be single or dual promoters. In a preferred embodiment, the promoter is the constitutively expressed ³⁵S promoter. In another preferred embodiment, the promoter is the inducible rap18 promoter. In yet another preferred embodiment, the promoter is the dual promoter mas1′2′ from the A. tumiefaciens Ti plasmid.

The nucleic acid sequence can further comprise any one or more of terminators, enhancers, promotors, or sequences to enable cloning and/or purification of the protein. These sequences include marker genes, poly restriction sites, and sequences that encode protein tags, such as FLAG, biotin, or polyhistidine tags. “Marker genes” include genes such as antibiotic resistance or sensitivity genes which may be used in identifying clones containing nucleic acid of interest, as is well known in the art.

The enhancers may for example be the sub-genomic transcript in the (Sgt) promoter (sequence −270 to +31 from the transcription start site; TSS), such as the Sgt of Figwort mosaic virus. The nucleic acid sequence can further comprise enhancer elements up-regulating plant enhancers, such as the tobacco Nuclear matrix attachment regions (MARs) sequences. Terminators comprised in the nucleic acid sequence may be classical terminators such as E9 from pea, ³⁵S from cauliflower mosaic virus (CaMV), polyAterminator. Furthermore, the nucleic acid sequence may further comprise sequences that can mediate termination from prokaryotic terminators such as thra, rrnB, rrnCand gene 32 terminators. An example could be the 3′-end of the spinach chloroplast (rbcL) gene.

In a second aspect the present invention provides a vector which contains the nucleic acid sequence defined aboved. This vector is, in one preferred embodiment C2200-DP-HK-LC.

In another aspect the present invention provides a host plant cell containing a nucleic acid construct, or vector as defined herein. A whole plant can be regenerated from the single transformed plant cell by procedures well known in the art. The invention also provides for propagating material or a seed comprising a cell. The invention also relates to any plant or part thereof including propagating material or a seed derived from any aspect of the invention.

In a preferred embodiment of the invention, the plant is a monocotyledoneous plant.

In another preferred embodiment of the invention, the plant is a dicotyledoneous plant.

In another preferred embodiment of the invention, the plant is an annual plant.

In another preferred embodiment of the invention, the plant is a biennial plant.

In another preferred embodiment of the invention, the plant is a perennial plant.

In a more preferred embodiment of the invention, the plant belongs to the Brassicaceae. In a further preferred embodiment of the invention the genetically modified plant belongs to the genus Arabidopsis.

In a most preferred embodiment of the invention, the plant belongs to the group consisting of the following species: Brassica napus, B. rapa, and B. junceaas, Brassica oleracea, Brassica napus, Brassica rapa, Raphanus sativus, Brassica juncea), Sinapis alba, Armoracia rusticana, Alliaria petiolata, Arabidopsis thaliana, A. griffithiana, A. lasiocarpa, A. petrea, Barbarea vulgaris, Berteroa incana, Brassica juncea, Brassica nigra, Brassica rapa, Bunias orientalis, Camelina alyssum, Camelina microcarpa, Camelina sativa, Capsella bursa-pastoris, Cardaria draba, Cardaria pubescens, Conringia orientalis, Descurainia incana, Descurainia pinnata, Descurainia sophia, Diplotaxis muralis, Diplotaxis tenuifolia, Erucastrum gallicum, Erysimum asperum, Erysimum cheiranthoides, Erysimum hieracifolium, Erysimum inconspicuum, Hesperis matronalis, Lepidium campestre, Lepidium densiflorum, Lepidium perfoliatum, Lepidium virginicum, Nasturtium officinale, Neslia paniculata, Raphanus raphanistrum, Rorippa austriaca, Rorippa sylvestris, Sinapis alba, Sinapis arvensis, Sisymbrium altissimum, Sisymbrium loeselii, Sisymbrium officinale, Thlaspi arvense, and Turritis glabra.

The plants according to the present invention preferably allow post-translational modifications and/or overproduction of the immunoglobulin proteins. This is preferably achived by the use of powerful transcription control sequences and antibody production may be further optimised by genetic engineering targeting the translational level or the posttranslational level.

The invention also provides a method of creating a library in a plant in which human antibodies can be expressed and arrayed against a panel of antigen targets. Such a technology may be desirable, as a major survival strategy of viruses such as HIV is to undergo genome mutations that lead to the modification of viral proteins. As a consequence, antibodies produced by infected patients become inefficient and the virus overcomes the immune response. By establishing individual plant lines expressing single or multiple antibodies directed against different viral antigen variants it will be possible to pool several lines to allow rapid selection of appropriate antibodies targeted against a given antigen using a grid-based screen. As an example a library of 10,000 different combinations could be screened such that the first layer of pooling 10 plants would reduce the pool number to 1,000, the second layer to 100 and the third one to 10 pools. Therefore each selection step would discard 90% of non-functional combinations. Based on this technique, from a library containing 10,000 combinations, only three selection steps would be sufficient to identify the pool of 10 plants that contains the specific antibody to be used for serotherapy or diagnostic.

Thus in a further aspect the present invention provides A method for selecting plants producing antibodies that bind to a specific protein, or fragment thereof, comprising the following steps:
(a) purify recombinant antibodies from a pool of plants expressing said antibodies;
(b) assay said antibodies to determine whether any bind to the specific protein or fragment thereof;
(c) and if the results of step (b) are positive, repeating steps (a) and (b) with the pool of plants sub divided into smaller groups; and
(d) repeating steps (a) to (c) until the plant producing the antibody that binds the specific protein or fragment thereof is identified.

In one preferrred embodiment antibodies are assyed by means of ELISA to identify those antibodies that bind to the specific protein or fragment thereof. The specific protein is preferably a viral protein, more preferably an HIV protein, most prefereably an HIV-1 envelope protein.

Hereinafter, the present invention will be described in detail by way of illustrative, but not restrictive, examples and with reference to the following figures:

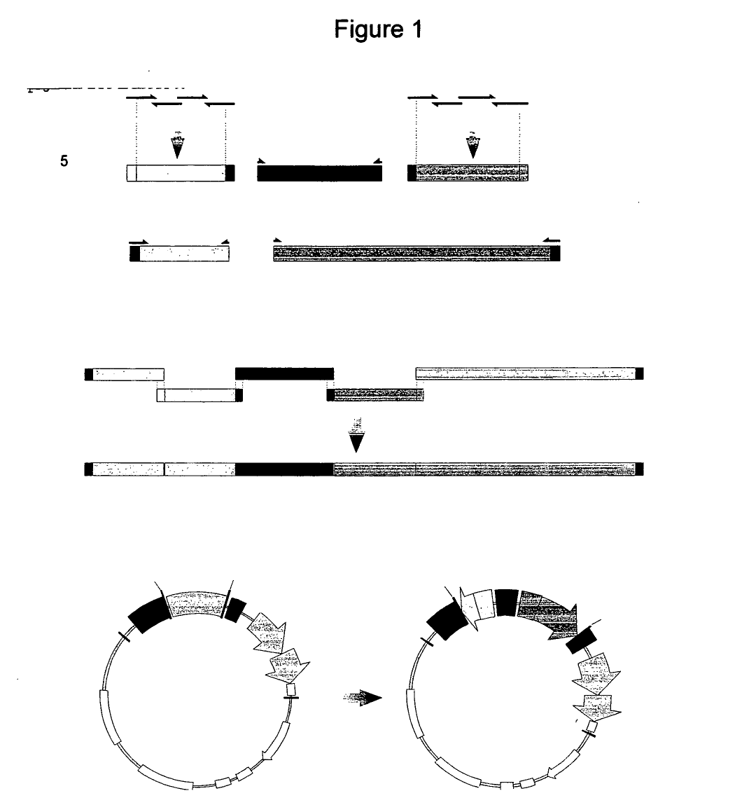

FIG. 1. Construction of a human antibody expression cassette targeted against different variants of the V3 loop of the HIV-1 virus envelope protein

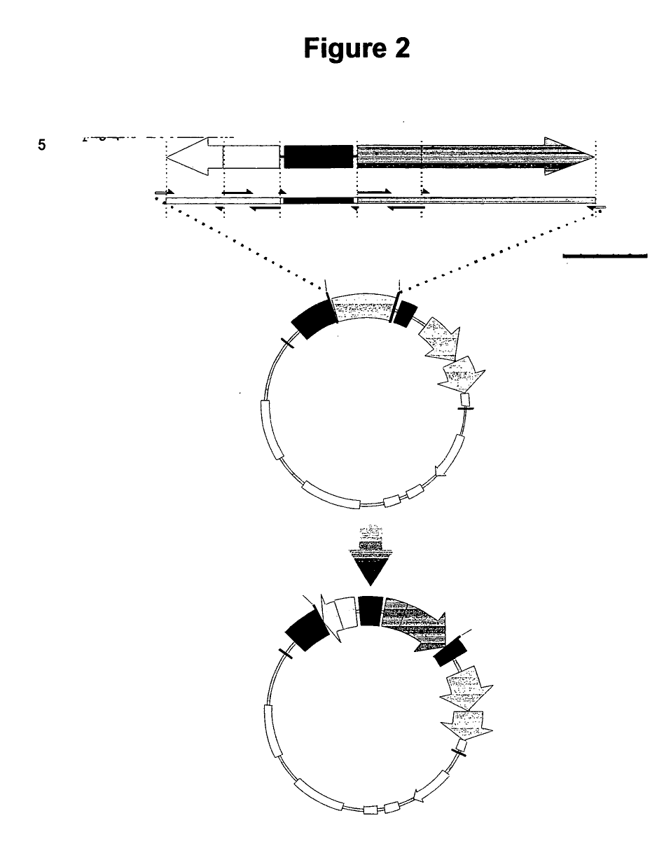

FIG. 2. Cloning of human antibody expression cassette into plasmid, capable of expressing antiboy in plants.

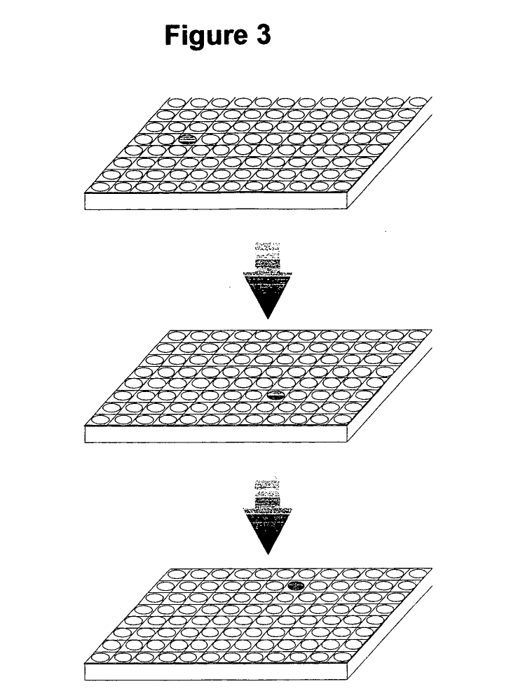

FIG. 3. Selection of transgenic lines by means of a grid based screening system

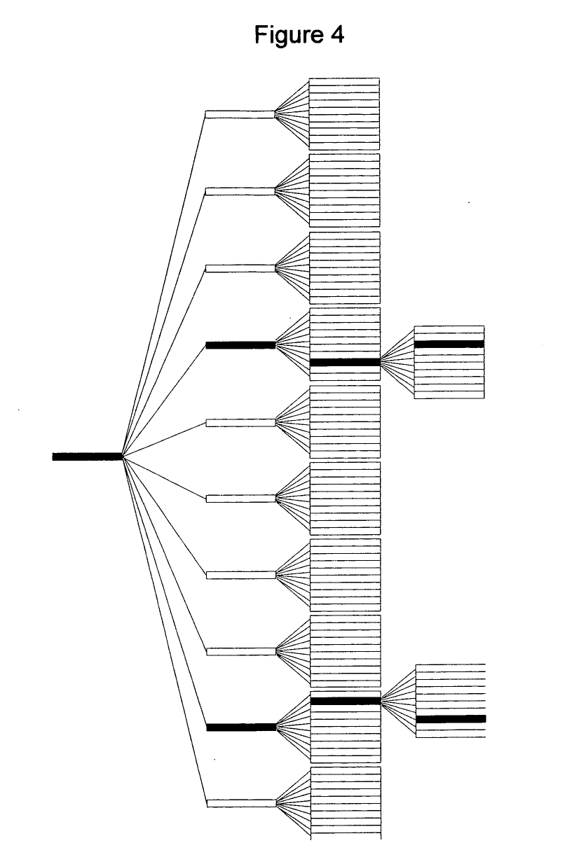

FIG. 4. Overview of pooling method of screening system.

### EXAMPLE 1

### Sequence Alignment of Human Antibody Light Chain cDNAs Targeted Against the Hypervariable V3 Loop of the HIV-1 virus gp120 Envelope Protein

Sequences were retrieved from the publicly available databases and aligned using a sequence alignment program. The sequence alignment of different human antibody light chain cDNAs targeted against the hypervariable V3 loop of HIV-1 was carried out. Alignment shows that the immunoglobulin chains exhibit well conserved sequences within the variable domains of antibodies. However, some 25 amino acid residues appear to be less conserved. This result suggests that these sequence differences reflect mutational variants of the viral protein.

### EXAMPLE 2

### Design of a Human Antibody Expression Cassette to be Used for Inserting the 25 Variable Motifs

A human antibody expression cassette containing both heavy chain (HC) and light chain (LC) genes coding for mABs targeted against the V3 loop of the HIV-1 virus gp120 envelope protein is described hereafter. Degenerated overlapping oligonucleotides representing different immunoglobulin variable heavy (VH) and variable light (VL) chain combinations were designed (Table 1). Each immunoglobulin variable chain is Polymerase Chain Reaction (PCR)-assembled independently by virtue of the complementarity between the degenerate primers used (FIG. 1). A second round of PCR is performed so that the VH and VL PCR fragments are fused to the mas1′2′ dual promoter (DP) from the A. tumefaciens Ti plasmid (Velten et al., 1984). This second round of PCR is performed in presence of DP DNA and the newly synthesized VH and VL PCR fragments, whose extremities close to the immunoglobulin ATG translational start codons are overlapping the DP fragment. The immunoglobulin constant heavy (CH) and constant light (CL) chain fragments are PCR-amplified independently in presence of CH and CL DNA template using a combination of forward and reverse oligonucleotides. The CH and CL PCR reverse primers were designed so that their 5′ regions respectively contain the attB1 and attB2 recombination sites, as well as unique restriction enzyme (RE) sites that can be used for sub-cloning the cassette. Using a PCR-based strategy, the CH and CL chain fragments are fused to the PCR fragment obtained in the second round of PCR by using CH and CL DNA templates whose upstream extremities are overlapping with the VH-DP-VL fragment. The subsequent cassette, here designed DP-HC-LC, containing HC and LC that are transcriptionally regulated by the DP promoter, and flanked by the unique RE sites and the attB recombination signals, is subsequently recombined or cloned into a plasmid (C2200-GATEWAY) that contains the attB sequences and two plant transcriptional terminators (FIG. 2). Additionally, this plasmid contains all features required for bacterial selection and plant transformation. The resulting plasmid, designated here C2200-DP-HC-LC is mobilized into E. coli bacteria using standard procedures to generate a library containing more than 10⁶ independent clones.

### EXAMPLE 3

### Transformation and Generation of 25 Arabidopsis thaliana Lines Expressing Human Antibodies Targeted against the V3 loop of the HIV-1 Virus Envelope Protein

For transformation of Arabidopsis thaliana, plants are grown to flowering stage under standard greenhouse conditions, 24° C. day/20° C. night. Plants are typically planted 20 per 64 cm² pot in moistened potting soil. To obtain more floral buds per plant, inflorescences are clipped after most plants have formed primary bolts to promote synchronized emergence of multiple secondary inflorescences. New inflorescences are allowed to grow for 8 days. Transgene constructs are mobilized into Agrobacterium tumefaciens by electroporation according to Shen and Forde (1989) prior to plant transformation by either vacuum infiltration (Bechtold and Pelletier, 1998) or by the floral dip method (Clough and Bent, 1998). After transformation, plants are allowed to self-pollinate, thereafter resulting T1-generation seedlings are selected on Murashige & Skoog Basal medium plates supplemented with 50 mg/l kanamycin, followed by transfer to soil. Approximately 20 lines for each construct are analyzed further.

Primary analyses are conducted on transgenic line to confirm expression of the immunoglobulin chains, correct folding of plantibodies, as well as functionality assays to evaluate binding to the antigens targets. These include RT-PCR and northern blot (to assess transgene expression), western blot under reducing and non-reducing conditions (to assess transgenic proteins production and correct immunoglobulin chains assembly). Antigen-antibody interaction assays are performed by means of enzyme-linked immunosorbent assay (ELISA) using HIV-1 envelope protein-coated immunoplates, followed by incubation with transgenic plant extract, and detection of the transgenic antibody using an alkaline phosphatase-conjugated secondary antibody.

### EXAMPLE 4

### Selection of Transgenic Lines by Means of a Grid-Based Screening Procedure

Transgenic plants expressing human antibodies targeted against the V3 loop of the HIV-1 virus envelope protein are allowed to self-pollinate. T₂-generation progeny seeds are mixed in pools of 1000 then plated on Murashige & Skoog Basal medium plates supplemented with 50 mg/l kanamycin. Recombinant antibodies are purified from each pool using standard techniques then each pool of antibodies is assayed for interaction with V3 loop variants of the HIV-1 virus envelope by means of sandwich ELISA assays. V3 loop variants are coated on 96-well flat-bottom plastic plates (Maxysorp, Nunc, Denmark) in 0.05 M carbonate/bicarbonate buffer, pH 9.6. (FIG. 3) Pools producing a positive results were subdivided into smaller pools and the process repeated to identify plants expressing the desired antibodies. (FIG. 4)

### REFERENCES

Belanger H, Fleysh N, Cox S, Bartman G, Deka D, Trudel M, Koprowski H, Yusibov V. Human respiratory syncytial virus vaccine antigen produced in plants. FASEB J. 2000 November;14 (14):2323-2328.
Bouquin T, Thomsen M, Nielsen L K, Green T H, Mundy J and Dziegiel M H (2002) Human anti-Rhesus D IgG1 antibody produced in transgenic plants. Transgenic Res 11: 115-122.
Chargelegue D, Vine N D, van Dolleweerd C J, Drake P M, Ma J K. A murine monoclonal antibody produced in transgenic plants with plant-specific glycans is not immunogenic in mice. Transgenic Res. 2000 June;9 (3):187-94.
De Jaeger G, De Wilde C, Eeckhout D, Fiers E, Depicker A. The plantibody approach: expression of antibody genes in plants to modulate plant metabolism or to obtain pathogen resistance. Plant Mol. Biol. 2000 July;43 (4):419-28.
Fischer R and Emans N (2000) Molecular farming of pharmaceutical proteins. Transgenic Res 9: 279-299.
Giddings G, Allison G, Brooks D and Carter A (2000) Transgenic plants as factories for biopharmaceuticals. Nat Biotechnol 18: 1151-1155.
Hiatt A, Cafferkey R and Bowdish K (1989) Production of antibodies in transgenic plants. Nature 342: 76-78.
Isaacs J D. The antiglobulin response to therapeutic antibodies. Seminars in Immunology 1990; 2: 449-456.
Kabat E A, Wu T T, Perry H M, et al. Sequences of proteins of immunological interest. US Department of Health and Human Services, U.S. Government Printing Office, 1991.
Kohler G, Milstein C. Continuous cultures of fused cells secreting antibody of predefined specificity. Nature 1975; 256: 495-497.
Ma J K, Hikmat B Y, Wycoff K, Vine N D, Chargelegue D, Yu L et al. (1998) Characterization of a recombinant plant monoclonal secretory antibody and preventive immunotherapy in humans. Nat Med 4: 601-606.
Richter L J, Thanavala Y, Arntzen C J, Mason H S. Production of hepatitis B surface antigen in transgenic plants for oral immunization. Nat Biotechnol. 2000 November;18 (11):1167-1171.
Verch T, Yusibov V and Koprowski H (1998) Expression and assembly of a full-length monoclonal antibody in plants using a plant virus vector. J Immunol Methods 220: 69-75.

Zeitlin L, Olmsted S S, Moench T R, Co M S, Martinell B J, Paradkar V M et al. (1998) A humanized monoclonal antibody produced in transgenic plants for immunoprotection of the vagina against genital herpes. Nat Biotechnol 16: 1361-1364.

TABLE 1

| Oligo- | | |
|---|---|---|
| nucleotide | Sequence (5′ → 3′) | Comment |
| DP1 | Ggagactgtgtcatcacgatgtc | Overlapping |
| | gggaatgcacactgtagctgttg | with VL-1 |
| | ctaccaagaagaggatgatacag | (bold) Murine |
| | ctccatcccatcttaagatcctt | leader (under- |
| | tatattgagatttttcaaatcag | lined) Start |
| | tgcgc | codon (bold & |
| | | underlined) |
| | | DP-specific |
| | | (normal font) |
| DP2 | Acgcctcccccagactgtaccag | Overlapping |
| | ctggacctggggaatgcacactg | with VH-1 |
| | tagctgttgctaccaagaagagg | (bold) Murine |
| | atgatacagctccatcccatcga | leader (under- |
| | tttggtgtatcgagattggttat | lined) Start |
| | gaaat | codon (bold & |
| | | underlined) |
| | | DP-specific |
| | | (normal font) |
| VH-1 | | |
| VH-2 | | |
| VH-3 | | |
| VL-1 | Gacatcgtgatgacacagtctcc | Overlapping |
| | agacaccctgtctttgtctccag | with DP-1 |
| | gggaaagagccaccctctcctgc | (bold) Over- |
| | agggccagtcagagtgttagcag | lapping with |
| | cggctacttagcctggtaccagc | VL-2 (under- |
| | agaaacctggccaggctcccagg | lined) |
| | ctcctcatctat | |
| VL-2 | Ctgacagtaatacactgcaaaat | Overlapping |
| | cttcaggctccagtctgctgatg | with VL-3 |
| | gtgagagtgaagtctgtcccaga | (bold) Over- |
| | cccactgccactgaacctgtctg | lapping with |
| | ggatgccagtggccctgctggag | VL-1 |
| | gcaccatagatgaggagcctggg | (underlined) |
| | agcctggccagg | |
| VL-3 | Cctgaagattttgcagtgtatta | Overlapping |
| | ctgtcagcagtatgatacctcac | with VL-2 |
| | ccgcgtggacgttcggccaaggg | (bold) |
| | accaaggtggaaatcaaacg | |

1. A nucleic acid construct comprising:
(i) a sequence encoding an immunoglobulin heavy chain,
(ii) a sequence encoding an immunoglobulin light chain, and
(iii) one or more promoters capable of controlling expression of both sequences in a plant,
wherein both said sequences contain a multivariable region in which preselected parts of said regions, which affect the encoded antibodies' ability to bind to variants of the target antigen, have been altered by exchanging, inserting or deleting one or more nucleotides as compared to the codes for the original immunoglobulin sequences.

2. The nucleic acid construct in accordance with claim 1, wherein the promoter in (iii) is a dual promoter.

3. The nucleic acid construct in accordance with claim 2, wherein said dual promoter is mas1′2′ from the A. tumiefaciens Ti plasmid.

4. The nucleic acid construct in accordance with claim 1 further comprising any one or more of the seuences selected from terminators, enhancers, promoters, and sequences to enable one of cloning and purification of the protein.

5. A nucleic acid construct in accordance with claim 1 that forms a vector.

6. A nucleic acid construct in accordance with claim 5 wherein the vector is C2200-DP-HC-LC.

7. A nucleic acid construct in accordance with claim 1 which is contained in a plant cell.

8. A nucleic acid construct in accordance with claim 7 contained in a plant cell that is contained in a plant part or in a whole plant.

9. A nucleic acid construct in accordance with claim 8 contained in a plant cell which is contained in a seed or other propagating material of a plant.

10. A method for the production of populations of antibodies comprising:
preparing a nucleic acid construct in accordance with claim 1 and expressing said nucleic acid construct in a plant.

11. A method according to claim 10 wherein said plants allows one of posttranslational modifications and overproduction of said immunoglobulin chains.

12. A method according to claim 11 comprising the step of post-translationally modifying the antibodies in the plants in vivo.

13. A method according to claim 11 comprising the step of post-translationally modifying the antibodies in the plants in vitro.

14. A method according to claim 10 further comprising a second method for selecting plants producing antibodies that bind to a specific protein, or fragment thereof, the second method comprising the following steps:
(a) purifying recombinant antibodies from a pool of plants expressing said antibodies;
(b) assaying said antibodies to determine whether any bind to the specific protein or fragment thereof;
(c) when positive binding in step (b) positive is found, repeating steps (a) and (b) with the pool of plants sub divided into smaller groups; and
(d) repeating steps (a) to (c) until the plant producing the antibody that binds the specific protein or fragment thereof is identified.

15. The method in accordance with claim 14 wherein the initial pool contains 1000 plants, which is subdivided by a factor of ten in step (c).

16. The method in accordance with claim 14 wherein the assay of step (b) is carried out by using a technique known as ELISA.

17. The method in accordance with claim 14 wherein the specific protein is a viral protein.

18. The method in accordance with claim 17 wherein the protein is an HIV virus protein.

19. The method in accordance with claim 18 wherein said protein is an HIV-1 envelope protein.

20. A pharmaceutical composition comprising an antibody identified by the method in accordance with claim 1.

21. (canceled)
